(19) United States
(12) Patent Application Publication
(10) Pub. No.: US 2022/0226063 A1
(43) Pub. Date: Jul. 21, 2022

(54) LASER POINTER DEVICE FOR USE IN A LAPROSCOPIC SURGERY PROCEDURE

(71) Applicant: UNIVERSIDADE DO MINHO, Braga (PT)

(72) Inventors: Pedro LEÃO, Braga (PT); Ana Cristina RIBEIRO, Braga (PT); Joao VILAÇA, Braga (PT); Nuno SOUSA, Braga (PT)

(21) Appl. No.: 17/609,524

(22) PCT Filed: May 8, 2020
(86) PCT No.: PCT/IB2020/054385

§ 371 Date: Nov. 8, 2021

(30) Foreign Application Priority Data
May 8, 2019 (PT) 115505
Mar. 23, 2020 (EP) 20165043.9

(51) Int. Cl. A61B 90/13

(52) U.S. Cl. CPC A61B 90/13

(57) ABSTRACT

A laser pointer device for use in a laparoscopic surgery procedure using an optical device is disclosed and has: a laser for pointing a laparoscopic surgery target; a fitting for attaching the laser to the optical device; and a user-operated controlled switch for remotely activating the laser pointer wherein the laser fitting is detachable from the optical device. The laser fitting can include at least one fixation point for attaching to the optical device. The switch can be arranged for being coupled to a handle of the optical device.

### TECHNICAL FIELD

The present disclosure relates to a laser pointer for clinical practice, in particular, a laser pointer for an optical device used in laparoscopic surgery procedures.

### BACKGROUND

The technological advances in the last decades have revolutionized the clinical practice and, in particular, of the general surgery. The introduction of minimally invasive surgery and its establishment as a standard procedure in many surgeries require knowledge and familiarity with new technologies and the development of new competencies both by residents and by already specialized surgeons who wish to adopt these technologies in their clinical practice. Increasing awareness of patients' rights and their safety, intolerance of wrongdoing and demands for medical-legal responsibility create new challenges for the traditional model of learning on the patients. To improve students' learning and minimize possible ethical issues, it is necessary to develop techniques that reduce the learning curve and the rate of avoidable errors.

There are already some solutions that allow to practice different gestures repeatedly and with different levels of complexity in an environment that is safe, controlled and adapted. The use of human cadavers and animal models has been established for centuries and allows a more realistic training. However, their availability is limited. The costs associated with the use of live animal models, the need for specialized assistants (such as veterinarians, anesthetists, etc.), ethical issues and animal rights policies, and health-related issues are also some of the barriers to its more widespread use.

Box training simulators (commonly known as endotrainers) allow you to practice basic techniques with real laparoscopic instruments and are essential for laparoscopic knot and suture training. However, they only allow the practice of specific techniques and do not allow the performance of whole procedures and the interaction with living and real tissues.

Virtual reality training simulators allow repeated practice of both basic procedures and entire surgeries in a virtual environment. In addition, the practitioner receives automatic instructions and feedback and their performance can be recorded in order to monitor their progress. However, in the light of current technology, these simulators still lack realism mainly because they do not allow a real interaction with the tissues nor produce a tactile sensation, besides being very expensive and little available.

Document WO9904737A1 discloses a laser powered surgical instrument that employs a target for transducing laser energy into shockwaves. The particular application of this invention is to a surgical instrument used in eye surgery and particularly for cataract removal. However, the document WO9904737A1 discloses an integrated laser pointer in devices which are adapted to surgical procedures.

Document EP1944054B1 discloses an invention can be used for a catheter, an examination system, and a thrombus removing device. However, the document EP1944054B1 discloses an integrated laser pointer in devices which are adapted to surgical procedures because the optical fiber is buried in a capillary tube, the optical fiber receiving a laser beam.

Thus, there is a need in the prior art for a device that reduces the learning curve and incidence of errors, in addition, being very economical and easily accessible and which can be used with sufficient accessibility in the operating room, shortening the time spent in surgical teaching, guidance and planning.

These facts are disclosed in order to illustrate the technical problem addressed by the present disclosure.

### GENERAL DESCRIPTION

The present disclosure aims to provide a laser pointer for a laparoscopic surgery optical device, usually used in laparoscopic surgery procedures.

This disclosure, the laser pointer, allows to demonstrate, in a real situation and patient, the route to be followed because the surgeon can point with the laser and in situ the exact planes that must be dissected. It has been already demonstrated in a surgical environment that the use of laser pointer reduces the learning curve and reduces the incidence of errors. In addition, it is very economical which makes it easily accessible and can be used with sufficient accessibility in the operating room, shortening the time spent in surgical planning, guidance and teaching.

The disclosure includes to pair a laser point to a normal optical device, usually used in laparoscopic surgery procedures with the switch located at the spot where the hand holds the optical device.

As already mentioned, this disclosure allows a surgeon, who is assisting in the surgical procedure, to hold the optics and, at the same time, to activate the laser and to directly show the anatomical structures and the planes that must be dissected, and identifying and pointing certain errors.

It is disclosed a laser pointer device for use in a laparoscopic surgery procedure using an optical device, comprising: a laser for pointing a laparoscopic surgery target; a laser fitting for attaching to the optical device; a user-operated controlled switch for remotely activating the laser pointer wherein the laser fitting is detachable from the optical device.

In an embodiment, the laser fitting can be comprised at least one fixation point for attaching to the optical device.

In an embodiment, the laser fitting is a cap for attaching to the optical device.

In an embodiment, the switch can be arranged for being coupled to a handle of the optical device.

In an embodiment, the laser pointer can comprise a battery pack for supplying power to the laser pointer device when activated.

In an embodiment, the laser pointer device can comprise a cable connection for connecting the battery pack to the laser.

In an embodiment, the laser pointer device can comprise a cable connection for connecting the user-operated controlled switch to the laser for remotely activating the laser.

In an embodiment, the laser pointer device can comprise a wireless connection for wirelessly connecting the user-operated controlled switch to the laser for remotely activating the laser.

In an embodiment, the battery pack can be arranged for being coupled to a handle of the optical device.

In an embodiment, the laser can be arranged to emit a red or green coloured beam.

In an embodiment, the laser pointer device is disposable.

In an embodiment, the case material of the laser pointer device is biocompatible.

It is also disclosed an article comprising the laser pointer device and said optical device, wherein the laser pointer device can be attached to the optical device by the laser fitting.

It is also disclosed a method for installing a laser pointer device for use in a laparoscopic surgery procedure using an optical device, comprising the following steps:

attaching the laser for pointing a laparoscopic surgery target by the laser fitting in the optical device.

It is also disclosed an article comprising a laser pointer device attached to the optical device by the described method.

### BRIEF DESCRIPTION OF THE DRAWINGS

The following figures provide preferred embodiments for illustrating the disclosure and should not be seen as limiting the scope of disclosure.

FIG. 1: Schematic conceptual representation of an embodiment of the laser pointer device paired to an optic device.

FIG. 2: Schematic conceptual representation of embodiments for three different laser fittings, in FIGS. 2a), 2b) and 2c), in particular comprising, respectively, an elastic band fitting, a tube-shaped fitting, a fitting comprising one or more rings.

FIG. 3: Schematic conceptual representation of embodiments for two different laser fittings, in FIGS. 3a) and 3b), in particular comprising, respectively, a cap-shaped fitting having a laser placed at the top of the cap and a cap-shaped fitting having a laser placed laterally along the cap.

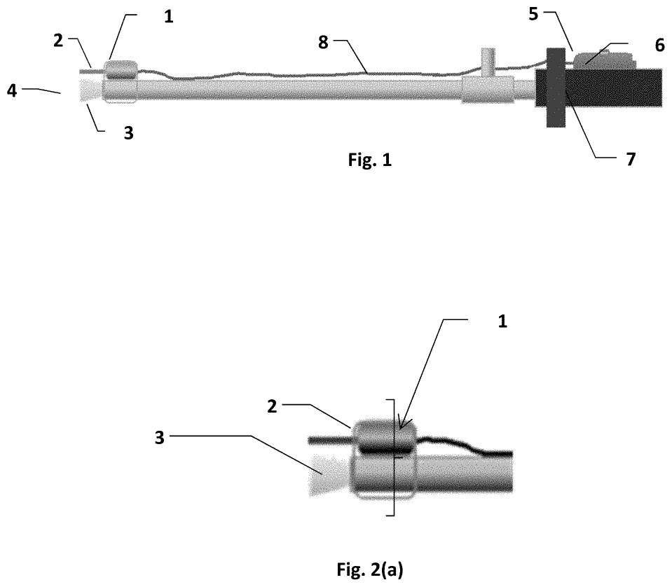

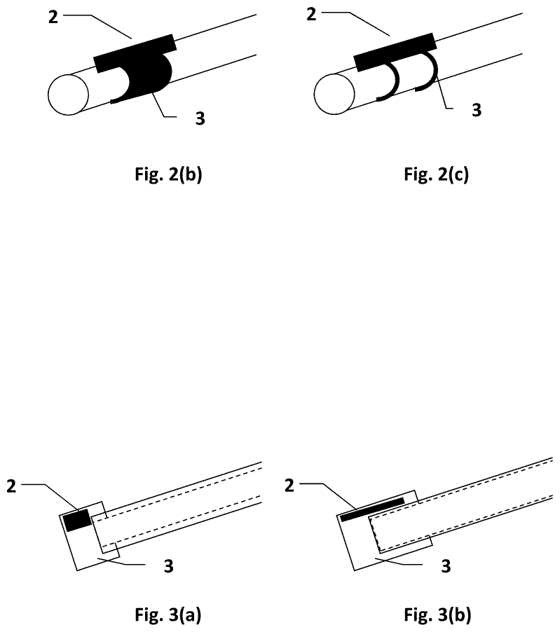

### DETAILED DESCRIPTION

The system according to an embodiment of the present disclosure is typically comprised by: the laser point (1) is paired to a normal optic device and se the switch is located at the place where the hand holds (2) the optic.

In an embodiment, the laser pointer device (1) for use in a laparoscopic surgery procedure using an optical device (4), comprising:
a laser (2) for pointing a laparoscopic surgery target;
a fitting (3) for attaching laser to the optical device (4); herewith referred to as a laser fitting;
a user-operated controlled switch (5) for remotely activating the laser (2);
wherein the laser fitting (3) is detachable from the optical device (4).

In an embodiment, the laser fitting (3) comprises at least one fixation point for attaching to the optical device (4).

In an embodiment, the laser fitting (3) is a cap for attaching to the optical device (4).

In an embodiment, the switch (5) is arranged for being coupled to a handle (7) of the optical device (4).

In an embodiment, the laser pointer device (1) comprises a battery pack (6) for supplying power to the laser pointer device (1) when activated and comprises a cable connection (8) for connecting the battery pack (6) to the laser (2).

In an embodiment, the laser pointer device (1) comprises a cable connection (8) for connecting the user-operated controlled switch (5) to the laser (2) for remotely activating the laser (2).

In an embodiment, the laser pointer device (1) comprises a wireless connection (8) for wirelessly connecting the user-operated controlled switch (5) to the laser (2) for remotely activating the laser (2).

In an embodiment, said battery pack (6) is arranged for being coupled to a handle of the optical device (4).

In an embodiment, the laser (2) is arranged to emit a red or green coloured beam.

In an embodiment, the device (4) is disposable.

In an embodiment, the laser fitting (3) is made of polypropylene, silicone, or their combinations.

In an embodiment, the laser fitting (3) comprises adhesive for attaching to the optical device (4).

This disclosure, the laser pointer, allows the tutor to demonstrate, in a real situation and patient, the route to be followed because the tutor can point with the laser and in situ the exact planes that must be dissected. It has already been demonstrated in a simulation environment that the use of laser pointer reduces the learning curve and reduces the incidence of errors. In addition, it is very economical which makes it easily accessible and can be used with sufficient accessibility in the operating room, shortening the time spent in surgical teaching.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable. The following claims further set out particular embodiments of the disclosure.

1. A laser pointer device for use in a laparoscopic surgery procedure using an optical device, comprising:
a laser for pointing a laparoscopic surgery target;
a fitting for attaching the laser to the optical device; and
a user-operated controlled switch for remotely activating the laser,
wherein the laser fitting is detachable from the optical device.

2. The laser pointer device according to claim 1, wherein the laser fitting comprises at least one fixation point for attaching to the optical device.

3. The laser pointer device according to claim 1, wherein the laser fitting is a cap for attaching to the optical device.

4. The laser pointer device according to claim 3, wherein the fitting has the laser placed at the top of the cap or the fitting has a cap-shaped fitting having the laser placed laterally along the cap.

5. The laser pointer device according to claim 1, wherein said switch is arranged for being coupled to a handle of the optical device.

6. The laser pointer device according to claim 1, wherein said laser pointer device comprises a battery pack for supplying power to the laser pointer device when activated.

7. The laser pointer device according to claim 6, wherein said laser pointer device comprises a cable connection for connecting the battery pack to the laser.

8. The laser pointer device according to claim 1, wherein said laser pointer device comprises a cable connection for connecting the user-operated controlled switch to the laser for remotely activating the laser.

9. The laser pointer device according to claim 1, wherein said laser pointer device comprises a wireless connection for wirelessly connecting the user-operated controlled switch to the laser for remotely activating the laser.

10. The laser pointer device according to claim 1, wherein said device is disposable.

11. The laser pointer device according to claim 1, wherein said the laser fitting is made of polypropylene, silicone, or their combinations.

12. The laser pointer device according claim 1, wherein said the laser fitting comprises adhesive for attaching to the optical device.

13. An article comprising the laser pointer device according to claim 1 configured for use for use in a laparoscopic surgery procedure, wherein the laser pointer device is attached to the optical device by the laser fitting.

14. A method for installing a laser pointer device according to claim 1 for use in a laparoscopic surgery procedure using an optical device, comprising the following step:
attaching the laser by the laser fitting to the optical device.

15. (canceled)
